(19) United States
(12) Patent Application Publication
(10) Pub. No.: US 2020/0129682 A1
(43) Pub. Date: Apr. 30, 2020

(54) SUPPORTER FOR BODY

(71) Applicants: Mitsui Chemicals, Inc., Tokyo (JP); KINESIO IP LLC, Albuquerque, NM (US)

(72) Inventors: Aya TAKEUCHI, Chiba (JP); Fumito TAKEUCHI, Chiba (JP); Tomoaki ITO, Chiba (JP); Kazuoki NAKAI, Chiba (JP); Kenzo KASE, Albuquerque, NM (US)

(21) Appl. No.: 16/176,160

(22) Filed: Oct. 31, 2018

(51) Int. Cl. A61M 1/08

(52) U.S. Cl. CPC A61M 1/08; A61M 2205/3606; A61M 2209/088; A61M 2205/0216

(57) ABSTRACT

A supporter for body (1) to be attached to a skin of a body includes a pad base member (10) which is made of an elastic member and has a curved shape at least locally, and an adhesion layer (11) which is provided in a surface of the pad base member (10) on a side being adhered to the skin. The adhesion layer (11) is adhered to the skin with the curved shape of the pad base member (10) being stretched along a shape of the body, and holds the skin in a state in which the skin is drawn up, using a restoring force of the pad base member (10).

### TECHNICAL FIELD

The present disclosure relates to a supporter for body.

### BACKGROUND ART

Conventionally known is cupping in which a cup is brought into intimate contact with each part of a body of a patient and sucking is performed, for the purpose of the patient's recovery from fatigue, or the other like purposes (refer to Patent Literature 1, for example).

### CITATION LIST

### Patent Literature

### PTL 1

### Japanese Utility Model Registration No. 3173362

### SUMMARY OF INVENTION

### Technical Problem

Meanwhile, with regard to cupping according to the background arts, while cupping is expected to change and encourage bloodstream in order to repair damaged tissue, congestion is entailed, which leads to internal hemorrhage in many cases. Further, some patients feel that stimulus or pain is too strong, or cannot withstand long-time use.

The present disclosure has been provided in view of the foregoing problem, and it is an object to provide a supporter for body (hereinafter called “body supporter”) which draws up a skin without giving a user pain or a strange feeling at a desired part of a body to promote circulation of body fluid under a skin more effectively, and is suitable for long-time use, and provide a method of attaching a body supporter.

### Solution to Problem

The present disclosure to solve the problem mentioned above provides a supporter for body to be attached to a skin of a body, the supporter including:

a pad base member made of an elastic member, the pad base member having a curved shape at least in part; and

an adhesion layer provided in a surface of the pad base member on a side to be adhered to the skin, wherein

the adhesion layer is adhered to the skin with the curved shape of the pad base member being stretched along a shape of the body, and the adhesion layer holds the skin in a state where the skin is drawn up, using a restoring three of the pad base member.

In another aspect, the present disclosure provides a method of attaching the supporter for body described above to a skin of a body, the method comprising:

adhering the adhesion layer to the skin while stretching the curved shape of the pad base member along a shape of the body; and

releasing an external force acting on the pad base member to allow the adhesion layer to hold the skin in a state in which the skin is drawn up, using a restoring force of the pad base member.

### BRIEF DESCRIPTION OF DRAWINGS

FIGS. 1A, 1B, and 1C are views showing a configuration of a body supporter according to Embodiment 1;

FIG. 2 is a perspective view of an appearance of the body supporter according to Embodiment 1;

FIG. 3 is a view showing a manner in which an adhesion layer of a pad base member according to Embodiment 1 is provided;

FIG. 4 is a view showing examples of an object to which the body supporter according to Embodiment 1 is applied;

FIG. 5 is a view for explaining a method of measuring a restoring force of the pad base member according to Embodiment 1;

FIG. 6 is a view for explaining the method of measuring a restoring force of the pad base member according to Embodiment 1;

FIG. 7 is a view for explaining a method of measuring stickiness of the adhesion layer according to Embodiment 1;

FIGS. 8A, 8B, 8C, and 8D are views for explaining a method of attaching a body supporter to a skin;

FIGS. 9A and 9B are views schematically showing change in a state under a skin of a body in a case where an adhesive tape is attached onto the skin;

FIG. 10 is a cross-sectional view of a side surface, showing a configuration of a body supporter according to Embodiment 2;

FIG. 11 is a cross-sectional view of a side surface, showing a configuration of a body supporter according to a modification of Embodiment 2;

FIGS. 12A and 12B are views showing a configuration of a body supporter according to Embodiment 3;

FIG. 13 is a cross-sectional view of a side surface, showing a configuration of a body supporter according to Embodiment 4;

FIG. 14 is a cross-sectional view of a side surface, showing a configuration of a body supporter according to Embodiment 5;

FIG. 15 is a cross-sectional view of a side surface, showing a configuration of a body supporter according to Embodiment 6;

FIG. 16 is a cross-sectional view of a side surface, showing a configuration of a body supporter according to Embodiment 7;

FIG. 17 is a cross-sectional view of a side surface, showing a configuration of a body supporter according to Embodiment 8; and

FIG. 18 is a cross-sectional view of a side surface, showing a configuration of a body supporter according to a modification of Embodiment 8.

### ADVANTAGEOUS EFFECTS OF INVENTION

The body supporter according to the present disclosure can more effectively draw up a skin without giving a user pain or a strange feeling at each of desired parts of a body.

### DESCRIPTION OF EMBODIMENTS

Hereinafter, preferred embodiments of the present disclosure will be described in detail with reference to accompanying drawings. It is noted that composing elements having substantially the same functions in the present specification and the drawings will be denoted by the same reference signs, so that duplicated description will be omitted.

### Embodiment 1

[Configuration of Body Supporter]

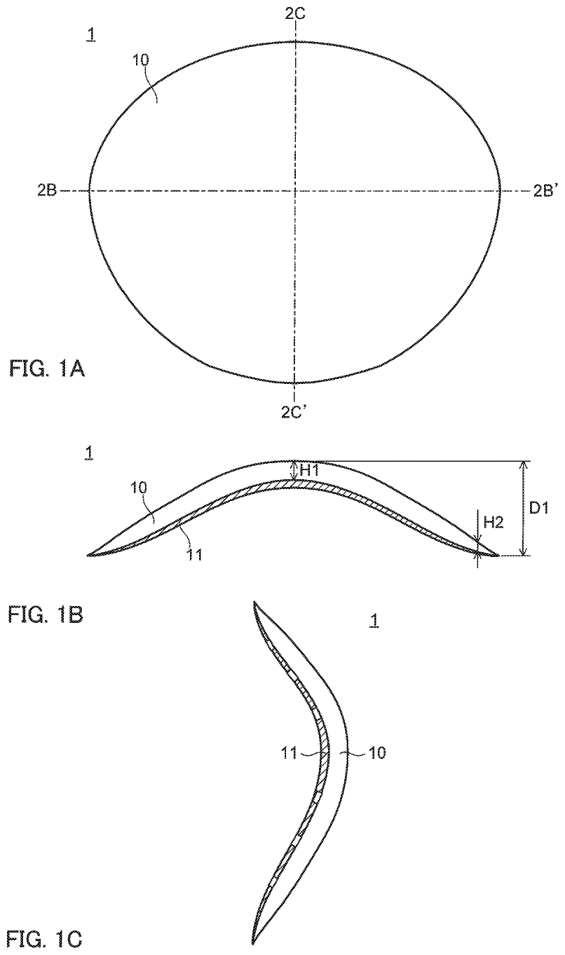

Below, one example of a configuration of a body supporter according to the present embodiment will be described with reference to FIGS. 1A to 7. In the present embodiment, body supporter 1 which is used in such a manner as to be attached to a waist of a human body as one example of an object to which body supporter 1 of the present invention is applied, will be shown.

FIGS. 1A to 1C, 2, and 3 are views showing a configuration of body supporter 1 according to the present embodiment.

FIG. 1A is a plan view of body supporter 1, FIG. 1B is a cross-sectional view of a side surface of body supporter 1, taken along a line 2B-2B′ in FIG. 1A, and FIG. 1C is a cross-sectional view of a side surface of body supporter 1, taken along a line 2C-2C′ in FIG. 1A.

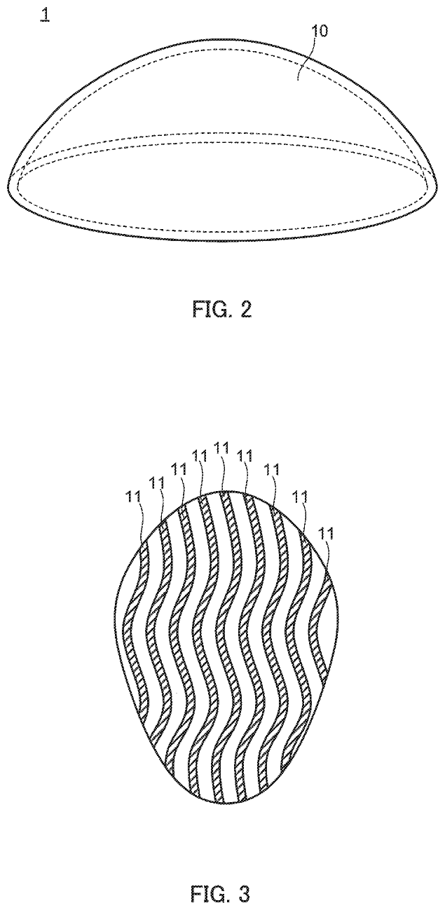

FIG. 2 is a perspective view of an appearance of body supporter 1. Moreover, FIG. 3 is a view showing a manner in which adhesion layer 11 of pad base member 10 is provided.

It is noted that in the following description, a direction of a top of a domed shape of pad base member 10 (upper side in FIG. 2) will be referred to as “upper” and a direction of a bottom surface of a domed shape of pad base member 10 (lower side in FIG. 2) will be referred to as “lower”, for convenience in description.

Body supporter 1 is made of an elastic member, and includes pad base member 10 which has a curved shape (a domed shape here) at least locally, and adhesion layer 11 which is provided in a surface of pad base member 10 on a side which is brought into contact with a skin.

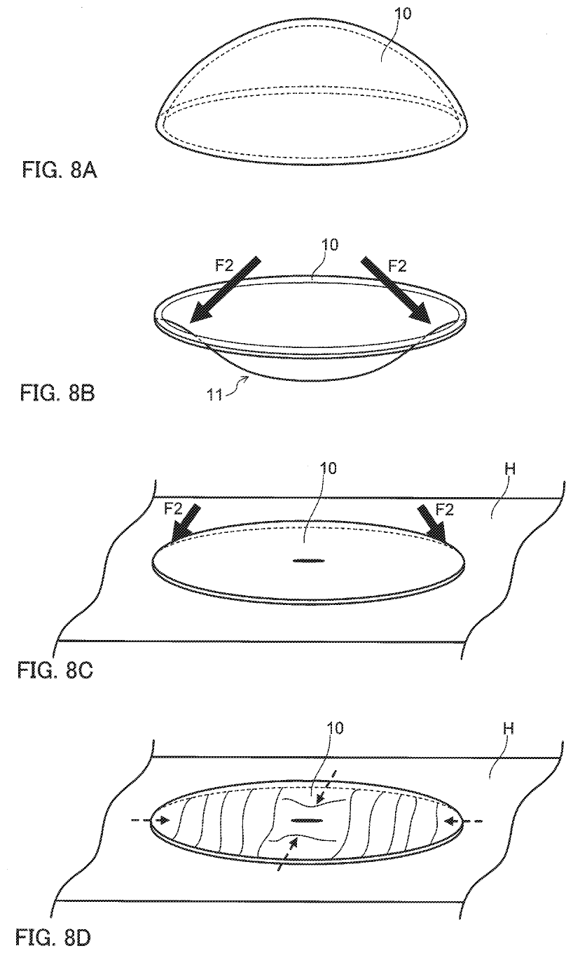

When body supporter 1 is attached to a skin, adhesion layer 11 is adhered to a skin of a body with a curved shape of pad base member 10 being changed along a shape of a body. Adhesion layer 11 adhered to a skin of a body is stretched along with transformation of pad base member 10. Then, in body supporter 1, when an external force exerted on pad base member 10 (typically, a tensile force exerted on pad base member 10 by hands, or an elastic force or a pressure which is generated by a press against a curved shape of pad base member 10 by hands) is released, pad base member 10 is restored and adhesion layer 11 is held in a state in which it is drawing up a skin (later described with reference to FIGS. 8A, 8B, 8C, and 8D).

It is noted that in body supporter 1, typically, a curved shape of pad base member 10 is changed to a nearly flat shape along a shape of a body, so that adhesion layer 11 is adhered to a skin of a body. In this regard, the term “nearly flat shape” means a shape of body supporter 1 or pad base member 10 which is transformed along a shape of a body in a position where it is attached, and includes an aspect in which a curved surface is locally included (the same shall apply hereinafter).

Pad base member 10 is a main part of body supporter

Pad base member 10 as a domed shape, typically. Thus, when pad base member 10 is transformed into a nearly flat shape and an external force exerted on pad base member 10 is released, pad base member 10 is restored from respective directions of an edge portion (representing an edge area of a domed shape with respect to a center of a domed shape assuming that a top of a domed shape corresponds to a center, and the same shall apply hereinafter) toward a central portion (representing an area around a center of a domed shape assuming that a top of a domed shape corresponds to a center, and the same shall apply hereinafter). That is, in this manner, body supporter 1 draws up a skin from respective directions of an edge portion toward a position of a central portion, via pad base member 10 and adhesion layer 11 (later described with reference to FIG. 8D).

Additionally it is preferable that a shape of pad base member 10 is a domed shape or a semi-cylindrical shape (including a semi-cylindrical shape with cross sections having different arc shapes in respective cut positions, and the same shall apply hereinafter) which is more concave than a part which is an attachment object in a body so that a skin of a part which is an attachment object in a body can be drawn up from respective directions. On the other hand, pad base member 10 may include a flat area in a part of a curved shape.

Any arbitrary material that is elastic can be used as a material of pad base member 10, and a urethan resin, a silicone resin, an elastomer resin, natural rubber, gel (physical gel, chemical gel), and the like are used, for example. Typically, a material of pad base member 10 is preferably chosen in view of a modulus of elasticity and flexibility (hardness), and pad base member 10 is typically in a gel form. Further, pad base member 10 is thrilled by an arbitrary method, and a resin molding method is used, for example.

It is preferable that a transparent material is used as a material of pad base member 10 in order to enable visual recognition of a attaching position and a attaching state. Further, in order to allow a user to attach body supporter 1 to a correct attaching position, a line indicating a position of a joint or a bone (an attaching-position guide or guideline) may be provided on a transparent pad base member, for example.

Pad base member 10 is typically made of a thin-film material having a thickness of 0.5 mm to 20 mm, inclusive, depending on what part is an attachment object in a body. However, it is preferable that a thickness of pad base member 10 decreases toward an edge portion from a central portion of pad base member 10. For example, it is preferable that thickness H1 of a central portion of pad base member 10 is equal to or greater than 1.5 times thickness H2 of an edge portion of pad base member 10, and is equal to or less than 150 times thickness H2. Thus, when body supporter 1 is attached, tensile stress acting on a skin in an edge portion of body supporter 1 can be relieved and a strange feeling caused in a body can be suppressed.

It is preferable that height D1 from a bottom surface of an edge portion of pad base member 10 to a top of a central portion of pad base member 10 is equal to or greater than 1 mm and is equal to or less than 50 mm when pad base member 10 is placed on a flat and horizontal test stand with adhesion layer 11 being located as a lower surface without application of an external force.

It is preferable that a diameter of curvature of an inner surface of a domed shape of pad base member 10 is equal to or greater than 15 mm and is equal to or less than 500 mm, depending on what part is an attachment object in a body. It is more preferable that the diameter of curvature is equal to or greater than 20 mm and is equal to or less than 450 mm, and it is much more preferable that the diameter of curvature is equal to or greater than 25 mm and is equal to or less than 400 mm. Additionally, respective curvatures along a major axis and a minor axis of pad base member 10 may be different from each other.

Adhesion layer 11 is provided in a whole of a surface of pad base member 10 on a side being adhered to a skin (lower surface), and holds adhesion between pad base member 10 and a skin. To form adhesion layer 11 in a whole of a lower surface of pad base member 10 makes it possible to draw up a skin entirely. However, adhesion layer 11 may be provided in a predetermined pattern in such a manner that a non-adhered area is provided locally in a lower surface of pad base member 10 as shown in FIG. 3. A pattern for adhesion layer 11 includes a pattern of dots, a pattern of stripes, a concentric pattern, a pattern of slits, a radial pattern, and the like, for example, so that alternating adhered and non-adhered areas are provided in a lower surface of pad base member 10.

As a result of formation of adhesion layer 11 in a lower surface of pad base member 10 in a predetermined pattern, a stress relieving area which suppresses concentration of tensile stress which is to act on a skin can be provided and a ventilation area can be provided locally between a skin and adhesion layer 11, when body supporter 1 is attached to a skin. Then, this can prevent a strange feeling from being caused by concentration of stress in a body, and allows water vapor emitted from a skin to escape outward. Further, to draw up a part of a skin in a predetermined pattern causes a skin to be drawn up while being wrinkled, and it is considered that this produces an effect of providing directionality to circulation of body fluid, or the like effect.

Besides, it is preferable that an area where adhesion layer 11 is formed occupies 30% to 100% of a whole area of a lower surface of pad base member 10 from the viewpoints of ensuring stickiness (adhesive force) required for adhesion layer 11 and ensuring good ventilation.

Any arbitrary material that ensures desired adhesion can be used as a material of adhesion layer 11, and a material of adhesion layer 11 is made of an adhesive agent such as an acrylic adhesive agent, a silicone-resin-based adhesive agent, or a rubber-based adhesive agent, for example. In addition, as adhesion layer 11, an alteration layer resulting from quality alteration of a lower surface of pad base member 10 may be used, or a carbon nanotube, a nanofiber, or the like which adheres to an object using Van der Waals force may be used.

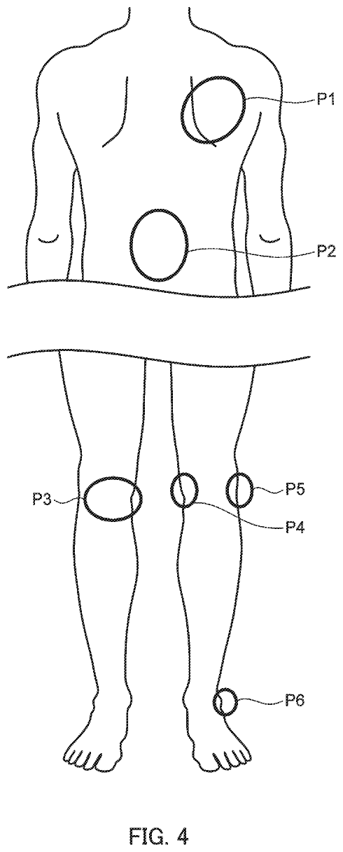

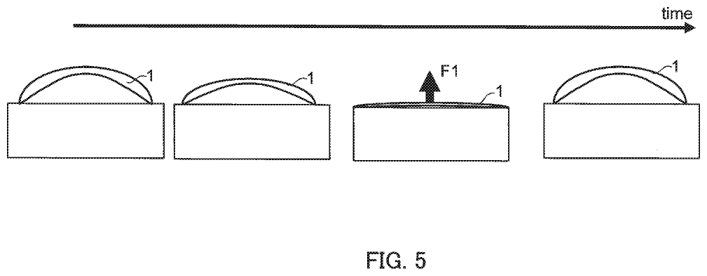

FIG. 4 is a view showing an example of an object to which body supporter according to the present embodiment is applied.

Body supporter 1 according to the present embodiment is applicable to shoulder P1, waist P2, front knee P3, inner knee P4, outer knee P5, ankle P6, and the like, for example.

It is noted that an oval area in each of shoulder P1, waist P2, front knee P3, inner knee P4, outer knee P5, and ankle P6 in FIG. 4 corresponds to an area where body supporter 1 is attached. However, a size and a shape of body supporter 1 can be appropriately changed depending on a user's request.

Now, desired values of a restoring force of pad base member 10 and stickiness (adhesive force) of adhesion layer 11 in body supporter 1 according to the present embodiment will be described.

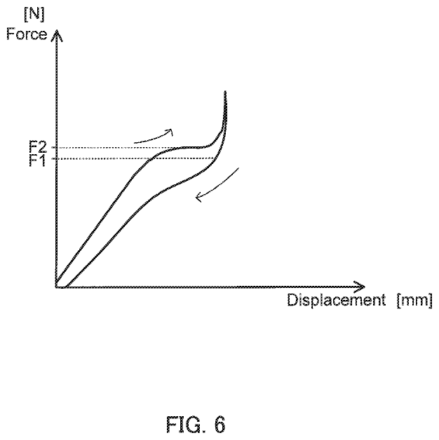

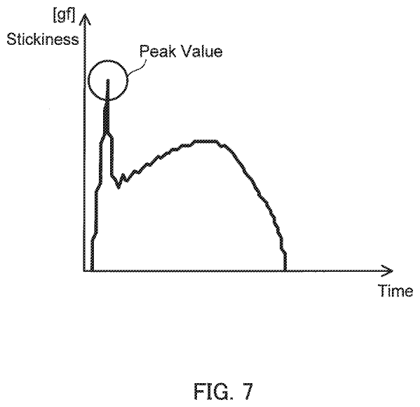

FIGS. 5 and 6 are views tier explaining a method of measuring a restoring force of pad base member 10 according to the present embodiment. In addition, FIG. 7 is a view for explaining a method of measuring stickiness of adhesion layer 11 according to the present embodiment.

It is preferable that body supporter 1 is so designed that adhesion layer 11 moderately draws up a skin when body supporter 1 is attached to a skin, from the viewpoint of effective promotion of circulation of body fluid under a skin, or the like. In other words, since body supporter 1 is kept attached to a skin for a long time, it is not preferable that a negative pressure is caused to act on a skin so strongly as to cause congestion or internal hemorrhage as in the case of cupping (cupping glass). On the other hand, it is also not preferable that an elastic force of body supporter 1 is too weak to draw up a skin when body supporter 1 is attached to a skin.

From the above-described viewpoints, it is preferable that a restoring force of pad base member 10 causes a tensile force which is equal to or greater than 0.01 N and is equal to or less than 15.0 N to act on a skin in a direction away from a skin, in response to release of an external force exerted on pad base member 10 when adhesion layer 11 is adhered to a skin. It is more preferable that a restoring force of pad base member 10 causes a tensile force which is equal to or greater than 0.05 N and is equal to or less than 12.5 N to act on a skin, and it is much more preferable that a restoring force of pad base member 10 causes a tensile force which is equal to or greater than 0.1 N and is equal to or less than 10.0 N to act on a skin.

It is noted that the above-described tensile force is set with respect to a measured. value of force F1 of pad base member 10 for returning upward when a top of a domed shape of pad base member 10 is pushed down and a curved shape of pad base member 10 is changed to a flat shape in a case where body supporter 1 is placed on a flat test stand with adhesion layer 11 being located as a lower surface as shown in FIG. 5, for example. More specifically; during a test, at a measuring temperature of 23° C., body supporter 1 is placed with a convex surface thereof up, and the highest portion is pushed down with a probe of 30 mmϕ at a speed of 10 mm/min. Then, pushing is continued until abrupt change in stress occurs due to a reaction force of a material used for body supporter 1, the reaction force being caused when the material is sandwiched between a bottom surface and the probe. From that point, a direction of the probe is changed, and the probe is moved at a speed of 10 mm/min in a direction away from body supporter 1. Then, the maximum value of a reaction force which is obtained when the probe is separated from body supporter 1 is defined as a “restoring force” (refer to FIG. 6). It is noted that stress applied to body supporter 1 moves as indicated by arrows in FIG. 6. F2 represents a maximum value of a pushing force and F1 represents a repulsion force.

Moreover, it is preferable that a restoring force of pad base member 10 causes a negative pressure which is equal to or higher than −5.50 kPa and is equal to or lower than −0.01 kPa to act on a skin in a direction in which a skin is drawn up, in response to release of an external force exerted on pad base member 10 when adhesion layer 11 is adhered to a skin. It is more preferable that a restoring force of pad base member 10 causes a negative pressure which is equal to or higher than −5.00 kPa and is equal to or lower than −0.05 kPa to act on a skin, and it is much more preferable that a restoring force of pad base member 10 causes a negative pressure which is equal to or higher than −4.50 kPa and is equal to or lower than −0.10 kPa to act on a skin.

Besides, the above-described negative pressure is set with respect to a measured value of a negative pressure acting on a flat test stand due to a restoring force of pad base member 10 when adhesion layer 11 is adhered onto the test stand with a curved shape being changed to a flat shape and an external force exerted on pad base member 10 is released. More specifically, during a test, a groove having a width of 2 mm is carved in a test stand (acrylic plate) having a thickness of 2 mm, and a terminal of a contact-pressure measuring apparatus (AMI3037-10 manufactured by AMI Techno CO., LTD.) is inserted into the groove, to fill a gap. The terminal is deflated beforehand using a syringe attached to the apparatus, and is connected to the apparatus after a release valve is opened. Then, body supporter 1 is naturally placed with a convex surface up so that a protrusion of body supporter 1 is located above the terminal, and a margin of approximately 5 mm is fixed while an opening is locally left. After body supporter 1 is pushed to be deflated as much as possible, the opening is blocked and a pressure is removed. Thereafter, measurement is conducted approximately five times while checking that air leakage does not occur and the probe is not pushed by a material, and a maximum value which is then obtained is defined as a “negative pressure”.

It is noted that a material, a thickness, a diameter of curvature, and the like of pad base member 10 are set such that a restoring force of pad base member 10 has a desired value which has been described above.

It is preferable that a peak value (i.e., a maximum instantaneous adhesive force) of stickiness (adhesive force) of adhesion layer 11 is equal to or greater than 40 gf and is equal to or less than 750 gf. Further, it is more preferable that the peak value is equal to or greater than 45 gf and is equal to or less than 700 gf, and it is much more preferable that the peak value is equal to or greater than 50 gf and is equal to or less than 650 gf. It is particularly preferable that the peak value is equal to or greater than 50 gf and is equal to or less than 300 gf. This can prevent a strange feeling from being given to a skin when adhesion layer 11 is adhered to a skin. Besides, the above-described stickiness of adhesion layer 11 is set based on a probe tack test, and a peak value of stickiness is measured as a peak value provided when adhesion layer 11 is peeled off an attachment object in a probe tack test as shown in FIG. 7. Specifically, the above-described probe tack test is conducted using a probe having a diameter of 5 mmϕ under conditions where a measuring temperature is 35° C., an approaching speed is 120 min/min, a pressurizing force is 30 gf, a pressurizing time is 60 seconds, and a separation speed is 600 min/min, to provide results. An average of ten points is used as a peak value (i.e., a maximum instantaneous adhesive force).

### EXAMPLES

Table 1 shows examples of pad base member 10 having the above-described restoring force. Each of Example 1, Example 2, Example 3, Example 4, and Example 5 is pad base member 10 having a domed shape. It is noted that pad base member 10 in Example 2 is vertically non-symmetrical and laterally non-symmetrical, and pad base member 10 of each of Example 1, Example 3, Example 4, and Example 5 is vertically non-symmetrical and laterally symmetrical.

### Comparative Examples

In comparative examples in Table 1, cupping (cupping glass made of a silicone resin, being distributed by Wakasugi enterprise Co., Ltd.) is used. Here, a small cupping glass is used in Comparative Example 1, a very small cupping glass is used in Comparative Example 2, and a soft-type pink cupping glass (small size) is used in Comparative Example 3.

A size in Table 1 results from measurement in which a sample is placed on a laboratory worktable or the like with a convex surface down and a 3D-scanner (HandySCAN 3D 700 manufactured by Creaform Inc.) is used. In this regard, attached software “VXelements” or the like is used. Based on thus obtained data, the longest axis is defined as a major axis and an axis extending in a direction approximately perpendicular to a major axis is defined as a minor axis, to calculate sizes. It is noted that in Comparative Examples 1 to 3, since a protrusion like a brim is included in a lower portion of a domed shape, an inner side is measured, avoiding the brim.

A height in Table 1 results from measurement in which a sample is placed on a laboratory worktable or the like with a convex surface down and the highest point is measured using Digimatic Height Gage HDS (570-322) manufactured by Mitutoyo Corporation. In Comparative Example 3, the neighborhood of a top of a domed shape except a protrusion for pushing is measured.

A thickness in Table 1 results from measurement in which ABS Digimatic Indicator ID-CX (543-400BS) manufactured by Mitutoyo Corporation, ABS Digimatic Thickness Gage (547-321) manufactured by Mitutoyo Corporation, and a probe of 10 mmϕ are used. Measurement is conducted three times using a portion with the largest thickness in each sample as a thickest portion and using a portion with the smallest thickness as a thinnest portion, and an average thereof is used.

A diameter of curvature in Table 1 results from calculation of diameters of curvature in which the longest axis is defined as a major axis and an axis extending in a direction approximately perpendicular to the major axis is defined as a minor axis based on the same data used for a size. However, calculation is performed regarding portions except a bottom surface because a central portion in each of the examples is pushed back by a bottom surface, to become flat. Besides, in Comparative Examples 1 to 3, since a protrusion like a brim is included in a lower portion of a domed shape, a portion inside the brim is measured.

A transforming force in Table 1 is one of factors in a sample's force of drawing up a skin. In Table 1, a force required to push down a center of a sample and flatten the sample is defined as a “pushing force”, and a force which is applied to a probe when a sample returns to its original shape from a flat shape is defined as a “restoring force”. It is noted that a maximum value for pushing down is employed as a “pushing force” in Table 1. Then, a may maximum value of a reaction force which is obtained when a probe is separated from a sample is employed as a “restoring force”. Additionally, a method of measuring a transforming force is as described above.

A negative pressure in Table 1 is one of indices of a sample's force of drawing up a skin. A method of measuring a negative pressure is as follows. It is noted that a method of measuring a negative pressure is as described above. Additionally, a measurement limit of a measurement apparatus is −8.60 kPa, and it is indicated that negative pressures are equal to or higher than the foregoing measurement limit in Comparative Examples 1 to 3.

Table 2 shows examples of stickiness of adhesion layer 11. In the table, an urethane resin “STABiO (registered trademark)” manufactured by Mitsui Chemicals, Inc., a silicone resin “KE-1051J” (silicone resin 1 in Table 2) manufactured by Shin-Etsu

Chemical Co., Ltd., a silicone resin “SILPURAN (registered trademark) 2117” (silicone resin 2 in Table 2) and a silicone resin “SILPURAN (registered trademark) 2142” (silicone resin 3 in Table 2) which are manufactured by Wacker Asahikasei Silicone Co., Ltd., and a silicone resin “KE-1056” (silicone resin 4 in Table 2) manufactured by Shin-Etsu Chemical Co., Ltd., are shown as examples of adhesion layer 11.

TABLE 1

| | | | | | | | | Comp. | Comp. | Comp. |
|---|---|---|---|---|---|---|---|---|---|---|
| | | | Example | Example | Example | Example | Example | Example | Example | Example |
| | | Unit | 1 | 2 | 3 | 4 | 5 | 1 | 2 | 3 |
| Size | Major | mm | 182.8 | 181.8 | 127.4 | 124.7 | 128.9 | 35.1 | 25.8 | 49.7 |
| | axis | | | | | | | | | |
| | Minor | mm | 149.3 | 140.3 | 109.2 | 107.7 | 108.4 | 34.9 | 25.8 | 49.5 |
| | axis | | | | | | | | | |
| | Height | mm | 43.55 | 45.76 | 30.45 | 29.64 | 28.12 | 22.07 | 16.06 | 32.16 |
| Thickness | Thickest | mm | 6.70 | 6.91 | 7.01 | 6.81 | 6.76 | 4.66 | 3.54 | 5.28 |
| | portion | | | | | | | | | |
| | Thinnest | mm | 3.30 | 3.60 | 2.33 | 1.70 | 0.69 | 4.02 | 3.19 | 4.73 |
| | portion | | | | | | | | | |
| | Thickest/ | — | 2.03 | 1.92 | 3.01 | 4.01 | 9.79 | 1.16 | 1.11 | 1.12 |
| | Thinnest | | | | | | | | | |
| Diameter of | Major | mm | 302.7 | 276.3 | 201.1 | 239.7 | 217.8 | 35.5 | 27.0 | 50.0 |
| curvature | axis | | | | | | | | | |
| | Minor | mm | 212.0 | 207.6 | 152.2 | 149.5 | 152.7 | 35.5 | 26.8 | 49.7 |
| | axis | | | | | | | | | |
| Diameter of | Major | — | 1.66 | 1.52 | 1.58 | 1.92 | 1.69 | 1.01 | 1.05 | 1.01 |
| curvature/Size | axis | | | | | | | | | |
| | Minor | — | 1.42 | 1.48 | 1.39 | 1.39 | 1.41 | 1.02 | 1.04 | 1.00 |
| | axis | | | | | | | | | |
| Transforming | Pushing | N | 1.38 | 2.40 | 5.84 | 5.51 | 4.37 | 54.51 | 135.45 | 34.20 |
| force | force | | | | | | | | | |
| | Restoring | N | 0.81 | 1.90 | 4.98 | 4.24 | 3.41 | 39.87 | 48.59 | 26.07 |
| | force | | | | | | | | | |
| Negative | | kPa | −1.73 | −1.68 | −4.19 | −3.42 | −2.26 | −8.60 | −8.60 | −8.60 |
| pressure | | | | | | | | | | |
| Negative | | kPa/mm | −0.0095 | −0.0092 | −0.0329 | −0.0274 | −0.0175 | −0.2450 | −0.3333 | −0.1730 |
| pressure/Size | | | | | | | | | | |
| (Major axis) | | | | | | | | | | |

TABLE 2

| | Peak value (gf) of |
|---|---|
| | stickiness |
| urethane resin | 652.8 |
| silicone resin 1 | 150.5 |
| silicone resin 2 | 192.5 |
| silicone resin 3 | 129.7 |
| silicone resin 4 | 68.8 |

[Method of Attaching Body Supporter]

Next, referring to FIGS. 8A to 8D, a method of attaching body supporter 1 to a skin will be described. It is noted that attaching of body supporter 1 according to the present embodiment is accomplished by human hands, for example.

FIGS. 8A to 8D are views for explaining a method of attaching body supporter 1 to a skin. FIGS. 8A to 8D show respective states of body supporter 1 in attaching body supporter 1 to a skin, in time series.

First, body supporter 1 is prepared (FIG. 8A). Subsequently, tensile force F2 (a pushing force, an elastic force, or a transforming force) is caused to act on pad base member 10 as an external force by human hands, so that a central portion of pad base member 10 is stretched (or pushed down, or leaned back) and a curved shape (domed shape) is changed to a nearly flat shape (FIG. 8B). It is noted that FIG. 8B shows an aspect in which a curved shape (domed shape) is changed to a nearly flat shape in such a manner that a central portion of pad base member 10 is pushed down, in order to facilitate attaching of body supporter 1 to a position where an attachment object is located.

Subsequently, while pad base member 10 is kept being transformed into a nearly flat shape, adhesion layer 11 is brought near to a desired portion in skin H of a body and adhesion layer 11 is adhered to skin H with pad base member 10 being transformed into a nearly flat shape along a shape of the body (FIG. 8C). Subsequently, external force F2 acting on pad base member 10 is released (FIG. 8D).

As a result of this, pad base member 10, which is kept being transformed into a nearly flat shape, is attached to skin H with adhesion layer 11 being interposed therebetween. Then, while pad base member 10 is in the process of restoration, pad base member 10 is held in a state in which a restoring force (an elastic force) of pad base member 10, an adhesive force of adhesion layer 11, and a reaction force exerted from skin H are balanced. Adhesion layer 11 holds skin H in a state in which skin H is drawn up toward a central portion of pad base member 10 from respective directions of an edge portion of pad base member 10 while pad base member 10 is in the process of restoration. At that time, skin H is drawn up while being wrinkled by adhesion layer 11 which is stretched along with transformation of the pad base member, in some cases.

It is noted that since body supporter 1 is stretchable and flexible, body supporter 1 follows motion of skin H while being attached to skin H even if a joint or the like of a body is folded after attaching.

[Effects]

As described above, body supporter 1 according to the present embodiment is made of an elastic member and includes pad base member 10 which has a curved shape at least locally and adhesion layer 11 which is provided in a surface of pad base member 10 on a side being adhered to a skin. Adhesion layer 11 is adhered to a skin with a curved shape of pad base member 10 being stretched, and holds a skin in a state where the skin is drawn up, using a restoring force of pad base member 10.

Accordingly, body supporter 1 according to the present embodiment allows a skin to be held in a state in which the skin is drawn up toward a central portion of pad base member 10 from respective directions of an edge portion of pad base member 10. This makes it possible to more effectively promote circulation of body fluid under a skin containing lymph, relieve pain, reduce distortion of tissue, or produce the other like effects.

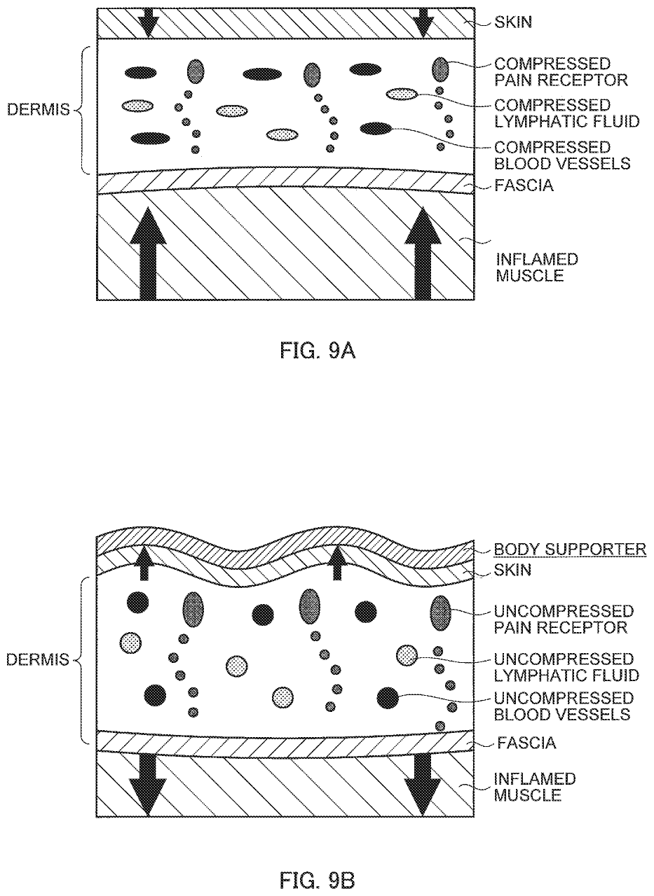

FIGS. 9A and 9B are views schematically showing a change in a state under a skin in a case where body supporter 1 according to the present embodiment is adhered onto a skin of a body. FIG. 9A shows a state before body supporter 1 according to the present embodiment is adhered onto a skin, and FIG. 9B shows a state after body supporter 1 according to the present embodiment is adhered onto a skin.

It is noted that in FIGS. 9A and 9B, muscle, fascia, dermis, and skin are shown sequentially from a lower-layer side.

Ordinarily, under a skin of a part at which pain or a strange feeling is given to a body, fascia or the like is strained and dermis containing a pain receptor, a blood vessel, and lymphatic fluid is compressed in most cases, as shown in FIG. 9A. Body supporter 1 according to the present embodiment is adhered to a skin while being stretched, and a skin is held while being drawn up using a restoring force of body supporter 1 according to the present embodiment, as shown in FIG. 9B. Thus, a gap is produced under a skin which is compressed.

As a result of this, circulation of body fluid (lymphatic fluid or tissue fluid, for example) is promoted, and effects of ease of pain, a kink, and internal hemorrhage, reduction of internal hemorrhage, relieve of pain in muscle, earlier recovery from muscle fatigue, rehabilitation, function restoration, improvement in motion of a joint, and the like, are expected.

### Embodiment 2

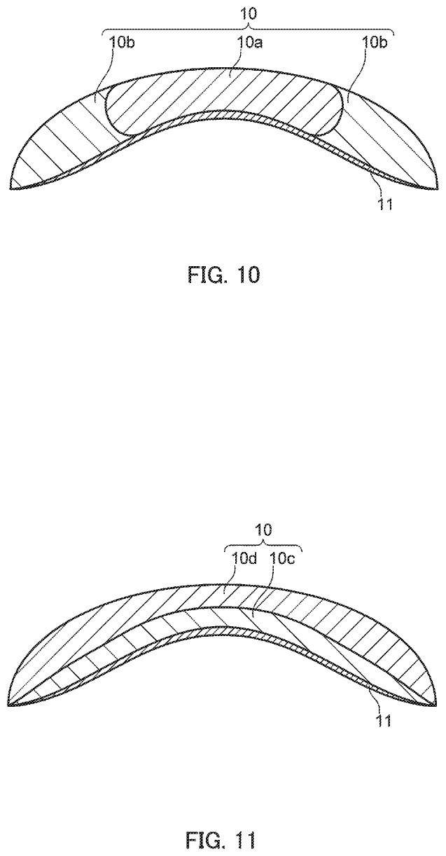

FIG. 10 is a cross-sectional view of a side surface, showing a configuration of body supporter 1 according to Embodiment 2.

Body supporter 1 according to the present embodiment is different from that of Embodiment 1 in that pad base member 10 includes a plurality of elastic materials having different moduli of elasticity. It is noted that description of elements which are the same as those in Embodiment 1 will be omitted (the same shall apply to the other embodiments).

Since body supporter 1 is to be attached to a skin, it is preferable that it is designed so as to reduce a strange feeling given to a body as much as possible. As main factors that give a strange feeling to a body, a modulus of elasticity of pad base member 10 and a pull of a skin by adhesion layer 11 are cited.

From this point of view, in body supporter 1 according to the present embodiment, elastic material 10a of a central portion of pad base member 10 is made of a material which has a higher modulus of elasticity and a greater restoring force than those of elastic material 10b of an edge portion of pad base member 10. Accordingly, an edge portion of pad base member 10 becomes soft, so that it is possible for a user to feel pleasant at a touch. Further, at that time, since a central portion of pad base member 10 is made of a material having a relatively high modulus of elasticity and a relatively great restoring force, also a tensile force acting on a skin can be sufficiently ensured by a restoring force of pad base member 10 when adhesion layer 11 is adhered to a skin.

FIG. 11 is a cross-sectional view of a side surface, showing a configuration of body supporter 1 according to a modification of Embodiment 2.

In body supporter 1 according to the present modification, elastic material 10d of pad base member 10 on a side close to adhesion layer 11 is made of a material which has a higher modulus of elasticity and a greater restoring force than those of elastic material 10c of pad base member 10 on a side opposite to adhesion layer 11.

Accordingly, a portion which comes in contact with a skin becomes soft, so that it is possible for a user to feel pleasant at a touch. Moreover, at that time, since an upper surface of pad base member 10 is made of a material having a relatively high modulus of elasticity and a relatively great restoring force, also a tensile force acting on a skin can be sufficiently ensured by a restoring force of pad base member 10 when adhesion layer 11 is adhered to a skin.

### Embodiment 3

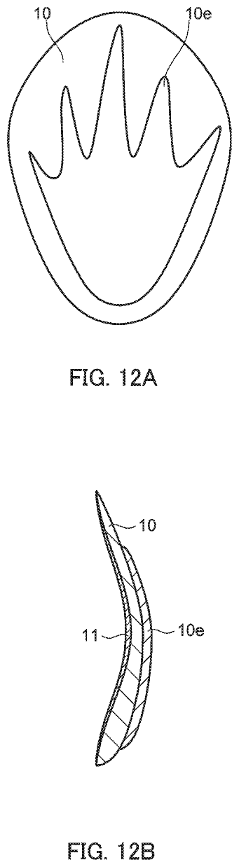

FIGS. 12A and 12B are views showing a configuration of body supporter 1 according to Embodiment 3. FIG. 12A is a plan view of body supporter 1 according to Embodiment 3, and FIG. 12B is a cross-sectional view of a side surface of body supporter 1 according to Embodiment 3.

Body supporter 1 according to the present embodiment is different from that of Embodiment 1 in that pad base member 10 includes skeleton member 10e.

Skeleton member 10e is provided in order to maintain an initial shape and an elastic force of pad base member 10, and has a curved shape (domed shape here) which is similar to a shape of pad base member 10 before transformation, for example. Skeleton member 10e is made of a material which has a higher modulus of elasticity than that of pad base member 10, and is made of a resin, gel, or metal, for example. Besides, it is preferable that skeleton member 10e has a shape of a palm in plan view, Thus, a strange feeling given to a body can be further suppressed and an effect similar to that produced by treatment can he expected.

### Embodiment 4

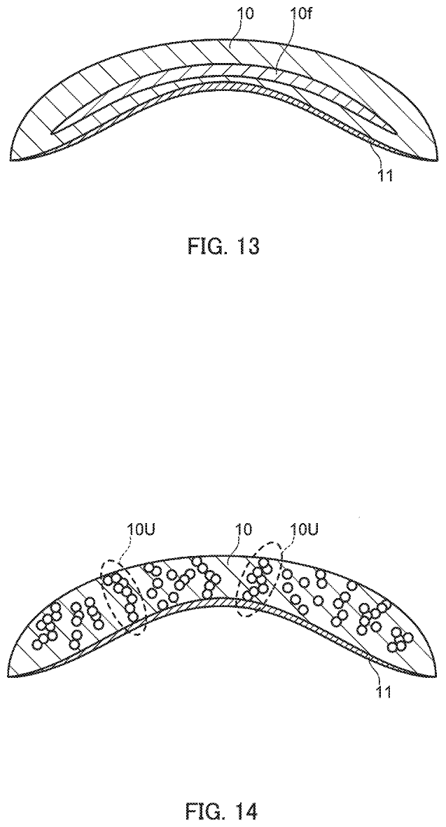

FIG. 13 is a cross-sectional view of a side surface, showing a configuration of body supporter 1 according to Embodiment 4.

Body supporter 1 according to the present embodiment is different from that of Embodiment 1 in that pad base member 10 includes material 10f which provides a function of providing a cool sensation at a touch, therein.

Material 10f which provides a function of providing a cool sensation at a touch is antifreeze gel, or a combination of water and a gelling agent, for example. Also, as material 10f which provides a function of providing a cool sensation at a touch, a material which reacts with water to cause endothermic reaction may be used, and material 10f is made of erythritol, xylitol (“PARACOOL ID-8”; Ohara Paragium Chemical Co., Ltd.), or the like, for example. Further, as the other materials, an ore of mica having high thermal conductivity and high hydratability may be used. A material having high thermal conductivity may be used. For example, metal is cited.

Also, in the other respects, it is preferable that material 10f which provides a function of providing a cool sensation at a touch is a material of which measurement value of a cool/humid sensation (q—max value) is equal to or higher than 0.2 in a state where the material is providing a function of providing a cool sensation at a touch.

However, a proportion of material 10f which performs a function of providing a cool sensation at a touch, to pad base member 10, is set such that a restoring force of pad base member 10 is not excessively impaired.

On the other hand, a material which performs a function of providing a warm sensation at a touch may be used in place of material 10f which provides a function of providing a cool sensation at a touch. In such a case, capsaicin, aluminum, tourmaline, a foam, and the like each of which has a heat insulation function may be used, for example.

Additionally; by using a microcapsule as material 10f which provides a function of providing a cool sensation at a touch or a material which performs a function of providing a warm sensation at a touch, it is possible to set a inciting point to a specific temperature through choice of a phase-change material contained in the microcapsule, and to extend a time during which the specific temperature is maintained. A heat/coolness storage agent (Miki Riken Industrial Co., Ltd., or Japan capsule products Co., Ltd.), a heat storage capsule (Mitsubishi Paper Mills Limited), and the like can be used as the foregoing microcapsule, for example.

As described above, body supporter 1 according to the present embodiment can give more comfort to a user.

### Embodiment 5

FIG. 14 is a cross-sectional view of a side surface, showing a configuration of body supporter 1 according to Embodiment 5.

Body supporter 1 according to the present embodiment is different from that of Embodiment 1 in that pad base member 10 includes communication hole 10U which allows a surface of pad base member 10 on a side close to adhesion layer 11 and a surface of pad base member 10 on a side opposite to adhesion layer 11 to communicate with each other.

Communication hole 10U provides good ventilation around adhesion layer 11 when adhesion layer 11 is adhered to a skin. Communication hole 10U can be formed by formation of pad base member 10 with the use of a porous material (urethane foam or silicon foam, for example), for example.

Additionally, it is preferable that body supporter 1 has moisture permeability which is equal to or greater than 1000 g/m² and ventilation resistance which is equal to or lower than 100 kPa·s/m when adhesion layer 11 is adhered to a skin.

As described above, body supporter 1 according to the present embodiment can give more comfort to a user.

### Embodiment 6

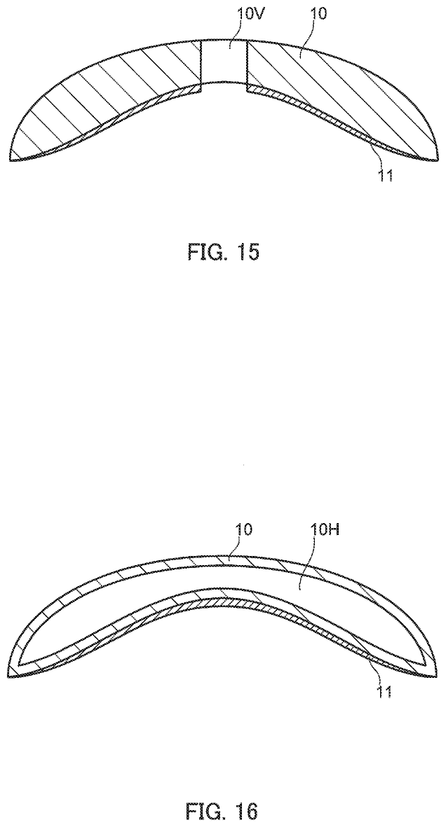

FIG. 15 is a cross-sectional view of a side surface, showing a configuration of body supporter 1 according to Embodiment 6. Body supporter 1 according to the present embodiment is different from that of Embodiment 1 in that pad base member 10 includes through hole 10V in a central portion of pad base member 10.

Through hole 10V allows a surface of pad base member 10 on a side close to adhesion layer 11 and a surface of pad base member 10 on a side opposite to adhesion layer 11 to communicate with each other in a central portion of pad base member 10.

Through hole 10V is particularly suitable in a case where a size of pad base member 10 is large, and also functions to relieve shear stress which acts on a skin when adhesion layer 11 is adhered to a skin. Further, through hole 10V functions to provide good ventilation and give more comfort to a user.

### Embodiment 7

FIG. 16 is a cross-sectional view of a side surface, showing a configuration of body supporter 1 according to Embodiment 7. Body supporter 1 according to the present embodiment is different from that of Embodiment 1 in that pad base member 10 includes hollow portion 10H.

Hollow portion 10H is formed in such a manner that an interior of pad base member 10 is locally hollowed out, for example. Thus, by providing hollow portion 10H in pad base member 10, it possible to lighten pad base member 10.

### Embodiment 8

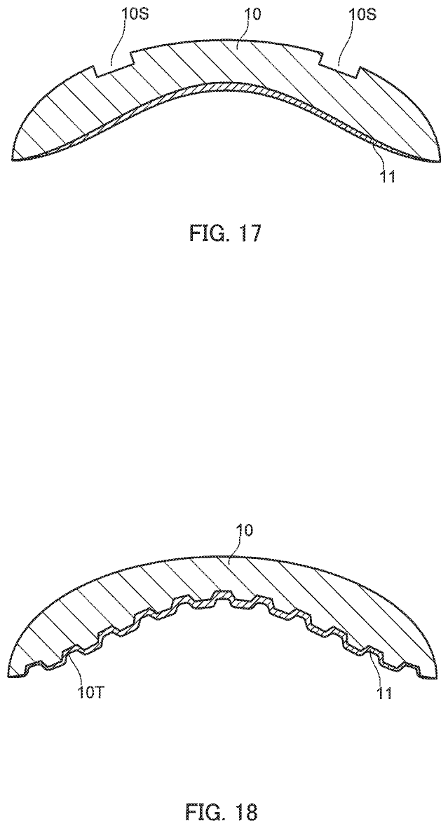

FIG. 17 is a cross-sectional view of a side surface, showing a configuration of body supporter 1 according to Embodiment 8. Body supporter 1 according to the present embodiment is different from that of Embodiment 1 in that uneven structure 10S is included in an upper surface of pad base member 10.

As shown, by providing uneven structure 10S in an upper surface of pad base member 10, it is possible to reduce a tensile force (a pushing force, an elastic force, or a transforming force) which serves as an external force required to manually stretch pad base member 10.

FIG. 18 is a cross-sectional view of a side surface, showing a configuration of body supporter 1 according to a modification of Embodiment 8. Body supporter 1 according to the present modification is configured so as to provide uneven structure 10T in a lower surface of pad base member 10.

As shown, also by providing uneven structure 10T in a lower surface of pad base member 10, it is possible to reduce a tensile force (a pushing force, an elastic force, or a transforming three) which serves as an external force required to manually stretch pad base member 10 in the same manner as in an aspect of FIG. 16.

As described above, body supporter 1 according to the present embodiment can reduce workload which is applied when body supporter 1 is attached to a skin of a body.

### Other Embodiments

The present invention is not limited to the above-described embodiments, and various modifications are conceivable.

For example, body supporter 1 may be formed integrally with a tape, a band, cloth, a supporter, clothes, or the like so that pad base member 10 is fixed, and may be used in a human body while being fixed to the foregoing objects beforehand.

Further, body supporter may be used after being cooled or heated beforehand.

Moreover, a guideline for fixing pad base member 10 to a predetermined position may be formed in an upper surface of pad base member 10.

Also, it is preferable that an upper surface of pad base member 10 has a coefficient of static friction which is equal to or lower than 0.9 so that pad base member 10 does not rub against a dress.

In addition, various combinations of aspects described above in the respective embodiments may be used as body supporter 1.

Hereinabove, though specific examples of the present invention have been described in detail, those have been described only for illustrative purposes and do not limit the claims. Arts recited in the claims include various alterations and modifications of the specific examples which have been illustratively described above.

### INDUSTRIAL APPLICABILITY

The supporter for body according to the present disclosure can more effectively promote circulation of body fluid under a skin, relieve pain, reduce distortion of tissue, or produce the other like effects.

### REFERENCE SIGNS LIST

1 Body supporter
10 Pad base member
11 Adhesion layer

1. A supporter for body to be attached to a skin of a body, the supporter comprising:
a pad base member made of an elastic member, the pad base member having a curved shape at least in part; and
an adhesion layer provided in a surface of the pad base member on a side to be adhered to the skin, wherein
the adhesion layer is adhered to the skin with the curved shape of the pad base member being stretched along a shape of the body, and the adhesion layer holds the skin in a state where the skin is drawn up, using a restoring force of the pad base member.

2. The supporter for body according to claim 1, wherein
the pad base member has a semi-cylindrical shape or a domed shape.

3. The supporter for body according to claim 2, wherein
a diameter of curvature of the pad base member is equal to or greater than 15 mm and is equal to or less than 500 mm.

4. The supporter for body according to claim 2, wherein,
in at least a part of the pad base member, a thickness of the pad base member includes a portion of the pad base member that is thinner than other portions.

5. The supporter for body according to claim 2, wherein
a thickness of the pad base member decreases toward an edge portion from a central portion of the pad base member.

6. The supporter for body according to claim 4, wherein
a thickness of a central portion of the pad base member is equal to or greater than 1.5 times a thickness of an edge portion of the pad base member and is equal to or less than 150 times the thickness of the edge portion of the pad base member.

7. The supporter for body according to claim 2, wherein
when the supporter is placed on a flat test stand with the adhesion layer being located as a lower surface without application of an external force, a height from a bottom surface of an edge portion of the pad base member to a top of a central portion of the pad base member is equal to or greater than 1 mm and is equal to or less than 50 mm.

8. The supporter for body according to claim 1, wherein
the adhesion layer is provided in a predetermined pattern in the surface of the pad base member on the side to be adhered to the skin.

9. The supporter for body according to claim 1, wherein
the restoring force of the pad base member causes a negative pressure that is equal to or higher than −5.50 kPa and is equal to or lower than −0.01 kPa to act on the skin in a direction in which the skin is drawn up, in response to release of an external force exerted on the pad base member when the adhesion layer is adhered to the skin.

10. The supporter for body according to claim 1, wherein
a peak value of stickiness of the adhesion layer equal to or greater than 40 gf and is equal to or less than 750 gf.

11. The supporter for body according to claim 10, wherein
the restoring force of the pad base member causes a tensile force that is equal to or greater than 0.01 N and is equal to or less than 15.0 N to act on the skin in a direction away from the skin in response to release of an external force exerted on the pad base member when the adhesion layer is adhered to the skin.

12. The supporter for body according to claim 1, wherein
the pad base member is made of a plurality of elastic materials having different moduli of elasticity.

13. The supporter for body according to claim 12, wherein
an elastic material of a central portion of the pad base member includes a material that has a higher modulus of elasticity and a higher restoring force than those of an elastic material of an edge portion of the pad base member.

14. The supporter for body according to claim 12, wherein
an elastic material of the pad base member on a side of the adhesion layer includes a material that has a higher modulus of elasticity and a higher restoring force than those of an elastic material of the pad base member on a side opposite to the adhesion layer.

15. The supporter for body according to claim 1, wherein
the pad base member includes a skeleton member having the curved shape.

16. The supporter for body according to claim 15, wherein
a material of the skeleton member is a material having a higher modulus of elasticity than that of the pad base member.

17. The supporter for body according to claim 1, wherein
the pad base member includes a communication hole that allows a surface of the pad base member on a side of the adhesion layer and a surface of the pad base member on a side opposite to the adhesion layer to communicate with each other.

18. The supporter for body according to claim 1, wherein
the pad base member includes a material that provides a function of providing a cool sensation at a touch.

19. The supporter for body according to claim 1, wherein
the pad base member includes a through hole in a central portion of the pad base member.

20. A method of attaching the supporter for body according to claim 1 to a skin of a body, the method comprising:
adhering the adhesion layer to the skin while stretching the curved shape of the pad base member along a shape of the body; and
releasing an external force acting on the pad base member to allow the adhesion layer to hold the skin in a state in which the skin is drawn up, using a restoring force of the pad base member.
